(19) United States
(12) Patent Application Publication
(10) Pub. No.: US 2022/0401366 A1
(43) Pub. Date: Dec. 22, 2022

(54) LONG ACTING NMDA ANTAGONISTS

(71) Applicant: Consegna Pharma, Inc., Pittsburgh, PA (US)

(72) Inventors: Lawrence Zana, Pittsburgh, PA (US); Stephen Hayward, Timonium, MD (US)

(21) Appl. No.: 17/776,691

(22) PCT Filed: Nov. 15, 2020
(86) PCT No.: PCT/US2020/070788

§ 371 Date: May 13, 2022

Related U.S. Application Data
(60) Provisional application No. 62/935,872, filed on Nov. 15, 2019.

(51) Int. Cl. A61K 9/16; A61K 47/44

(52) U.S. Cl. CPC A61K 9/1647; A61K 9/1617; A61K 47/44

(57) ABSTRACT

Sustained release formulations of NMDA antagonists containing encapsulated NMDA antagonist are described herein.

### CROSS-REFERENCE TO RELATED APPLICATIONS

This application claims priority from U.S. Provisional No. 62/935,872 entitled “Long Acting NMDA Antagonists” filed on Nov. 15, 2019, the entire contents of which is hereby incorporated by reference.

### GOVERNMENT INTERESTS

Not applicable

### PARTIES TO A JOINT RESEARCH AGREEMENT

Not applicable

### INCORPORATION OF MATERIAL ON COMPACT DISC

Not applicable

### BACKGROUND

Low doses of the NMDA antagonists such as ketamine have been shown to provide antidepressant effects in patients with various neuropsychiatric disorders including treatment-resistant anxiety (TRA) disorders, Post-Traumatic Stress Disorder (PTSD), Obsessive Compulsive Disorder (OCD), Generalized Anxiety Disorder (GAD), and Social Anxiety Disorder (SAD. Patients receiving NMDA treatment are generally injected with the NDMA antagonist, e.g. ketamine, intravenously. However, ketamine injection can result in dissociative symptoms that occur mainly in the first hour after dosing and minor increases in blood pressure and heart rate, which occur in the first 30 minutes. Long acting formulations of NMDA antagonists may reduce such side effects, making the NMDA treatment a better alternative for neuropsychiatric patients.

### SUMMARY OF THE INVENTION

Various embodiments of the invention are directed to compositions containing about 100 mg to about 500 mg of NMDA antagonist encapsulated in microparticles and a pharmaceutically acceptable excipient, diluent, or carrier. In such embodiments, the NMDA antagonist may be minocycline, amantadine, atomoxetine, AZD6765, agmatine, chloroform, dextrallorphan, dextromethorphan, dextrorphan, diphenidine, dizocilpine (MK-801), ethanol, eticyclidine, gacyclidine, ketamine, magnesium, memantine, methoxetamine, nitromemantine, nitrous oxide, PD-137889, phencyclidine, rolicyclidine, methoxydine, tiletamine, neramexane, eliprodil, etoxadrol, dexoxadrol, WMS-2539, NEFA, remacemide, delucemine, 8A-PDHQ, and the like and combinations thereof. In particular embodiments, the NMDA antagonist may be ketamine (2-(2-chlorophenyl)-2-(methylamino)-cyclohexanone), S-ketamine, esketamine, R-ketamine, racemic ketamine, norketamine (2-(2-chlorophenyl)-2-amino-cyclohexanone), S-norketamine, R-norketamine, and racemic norketamine.

In some embodiments, the microparticles may be composed of a biodegradable polymer such as, for example, polylactides (PLA), polyglycolides (PGA), poly(lactide-co-glycolide) (PLGA) polymers, poly butylene succinate (PBS), polyhydroxyalkanoate (PHA), polycaprolactone acid lactone (PCL), polyhydroxybutyrate (PHB), glycolic amyl (PHV), PHB and PHV copolymer (PHBV), and poly lactic acid (PLA)-polyethylene glycol (PEG) copolymers (PLEG), and copolymers thereof. In certain embodiments, the poly(lactide-co-glycolide) (PLGA) may be capped, and in some embodiments, the poly(lactide-co-glycolide) (PLGA) may have a ratio of polylactides (PLA) to polyglycolides (PGA) of 50:50 by weight to about 60:40 by weight. In particular embodiments, the microparticles may have a mean particle diameter of about 40 μm to about 60 μm.

The compositions of embodiments may further include methyl cellulose (MC), ethyl cellulose (EC), ethyl methyl cellulose (EMC), hydroxyethyl cellulose (HEC), hydroxylpropyl cellulose (HPC), hydroxymethyl cellulose (HMC), hydroxypropylmethyl cellulose (HPMC), ethylhydroxyethyl cellulose (EHEC), hydroxyethylmethy cellulose (HEMC), methylhydroxyethyl cellulose (MHEC), methylhydroxypropylcellulose (MHPC), hydroxyethylcarboxymethyl cellulose (HECMC), and the like and combinations thereof. In some embodiments, the excipient may be calcium carbonate, calcium phosphate, various sugars, starches, cellulose derivatives, gelatin, polyethylene glycol, and combinations thereof. In some embodiments, the compositions may further include a binder, coating, disintegrant, filler, diluent, flavor, color, lubricant, glidant, preservative, sorbent, sweetener, conjugated linoleic acid (CLA), gelatin, beeswax, purified water, glycerol, pharmaceutically acceptable oils, and the like and combinations thereof.

Some embodiments include a composition containing about 100 mg to about 500 mg of NMDA antagonist and a pharmaceutically acceptable oil. In various embodiments, the pharmaceutically acceptable oil may be, for example, vegetable oil, olive oil, grapeseed oil, tea tree oil, almond oil, avocado oil, sesame oil, evening primrose oil, sunflower oil, kukui nut oil, jojoba oil, walnut oil, peanut oil, pecan oil, macadamia nut oil, coconut oil, or combinations thereof. In certain embodiments, the NMDA antagonist may be ionically associated with the pharmaceutically acceptable oil. The NMDA antagonist of such embodiments may be iminocycline, amantadine, atomoxetine, AZD6765, agmatine, chloroform, dextrallorphan, dextromethorphan, dextrorphan, diphenidine, dizocilpine (MK-801), ethanol, eticyclidine, gacyclidine, ketamine, magnesium, memantine, methoxetamine, nitromemantine, nitrous oxide, PD-137889, phencyclidine, rolicyclidine, methoxydine, tiletamine, neramexane, eliprodil, etoxadrol, dexoxadrol, WMS-2539, NEFA, remacemide, delucemine, 8A-PDHQ, and the like and combinations thereof.

Various other embodiments are directed to methods for treating neuropsychiatric disorders by administering to a subject in need of treatment a composition about 100 mg to about 500 mg of NMDA antagonist encapsulated in microparticles and a pharmaceutically acceptable excipient, diluent, or carrier wherein the composition releases the NMDA antagonist for about 7 days to about 90 days. In some embodiments, administering can be carried out parenteral injection, subcutaneous injection, intravenous injection, intramuscular injection, intraperitoneal injection, transdermally, orally, buccally, ocularly, intravaginally, by inhalation, by depot injections, or by implants, and in certain embodiments, administering can be carried out by depo injection, intramuscular injection, or subcutaneous injection.

The method of claim 15, wherein the NMDA antagonist is selected from the group consisting of minocycline, amantadine, atomoxetine, AZD6765, agmatine, chloroform, dextrallorphan, dextromethorphan, dextrorphan, diphenidine, dizocilpine (MK-801), ethanol, eticyclidine, gacyclidine, ketamine, magnesium, memantine, methoxetamine, nitromemantine, nitrous oxide, PD-137889, phencyclidine, rolicyclidine, methoxydine, tiletamine, neramexane, eliprodil, etoxadrol, dexoxadrol, WMS-2539, NEFA, remacemide, delucemine, 8A-PDHQ, and combinations thereof.

In such embodiments, the NMDA antagonist may be minocycline, amantadine, atomoxetine, AZD6765, agmatine, chloroform, dextrallorphan, dextromethorphan, dextrorphan, diphenidine, dizocilpine (MK-801), ethanol, eticyclidine, gacyclidine, ketamine, magnesium, memantine, methoxetamine, nitromemantine, nitrous oxide, PD-137889, phencyclidine, rolicyclidine, methoxydine, tiletamine, neramexane, eliprodil, etoxadrol, dexoxadrol, WMS-2539, NEFA, remacemide, delucemine, 8A-PDHQ, and the like and combinations thereof. In particular embodiments, the NMDA antagonist may be ketamine (2-(2-chlorophenyl)-2-(methylamino)-cyclohexanone), S-ketamine, esketamine, R-ketamine, racemic ketamine, norketamine (2-(2-chlorophenyl)-2-amino-cyclohexanone), S-norketamine, R-norketamine, and racemic norketamine.

In some embodiments, the microparticles may be composed of a biodegradable polymer such as, for example, polylactides (PLA), polyglycolides (PGA), poly(lactide-co-glycolide) (PLGA) polymers, poly butylene succinate (PBS), polyhydroxyalkanoate (PHA), polycaprolactone acid lactone (PCL), polyhydroxybutyrate (PHB), glycolic amyl (PHV), PHB and PHV copolymer (PHBV), and poly lactic acid (PLA)-polyethylene glycol (PEG) copolymers (PLEG), and copolymers thereof. In certain embodiments, the poly(lactide-co-glycolide) (PLGA) may be capped, and in some embodiments, the poly(lactide-co-glycolide) (PLGA) may have a ratio of polylactides (PLA) to polyglycolides (PGA) of 50:50 by weight to about 60:40 by weight. In particular embodiments, the microparticles may have a mean particle diameter of about 40 μm to about 60 μm.

The compositions of embodiments may further include methyl cellulose (MC), ethyl cellulose (EC), ethyl methyl cellulose (EMC), hydroxyethyl cellulose (HEC), hydroxylpropyl cellulose (HPC), hydroxymethyl cellulose (HMC), hydroxypropylmethyl cellulose (HPMC), ethylhydroxyethyl cellulose (EHEC), hydroxyethylmethy cellulose (HEMC), methylhydroxyethyl cellulose (MHEC), methylhydroxypropylcellulose (MHPC), hydroxyethylcarboxymethyl cellulose (HECMC), and the like and combinations thereof. In some embodiments, the excipient may be calcium carbonate, calcium phosphate, various sugars, starches, cellulose derivatives, gelatin, polyethylene glycol, and combinations thereof. In some embodiments, the compositions may further include a binder, coating, disintegrant, filler, diluent, flavor, color, lubricant, glidant, preservative, sorbent, sweetener, conjugated linoleic acid (CLA), gelatin, beeswax, purified water, glycerol, pharmaceutically acceptable oils, and the like and combinations thereof.

### DESCRIPTION OF THE DRAWINGS

For a fuller understanding of the nature and advantages of the invention, reference should be made to the following detailed description taken in connection with the accompanying drawings, in which:

FIG. 1 is a graph showing the in vitro cumulative release of norketamine-HCl for several formulations.

FIG. 2 is a graph showing the predicted PK profile for each of the formulations tested in FIG. 1.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 ml to 8 ml is stated, 2 ml, 3 ml, 4 ml, 5 ml, 6 ml, and 7 ml are also intended to be explicitly disclosed, as well as the range of values greater than or equal to 1 ml and the range of values less than or equal to 8 ml and non-integers such as 2.5 ml, 4.33 ml, 5.25 ml, 6.75 ml, and the like.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The terms “effective amount” and “therapeutically effective amount” are used interchangeably in this disclosure and refer to an amount of a compound that, when administered to a subject, is capable of reducing a symptom of a disorder in a subject or enhance the texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g., animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments of the invention are directed to pharmaceutical compositions for sustained release of NMDA antagonists, over a period of up to about 60 days and, in some embodiments, about 5 days to about 40 days or about 7 days to about 30 days, about 10 days to about 30 days, about 10 days to about 21 days, or any range or individual time period encompassed by these examples. Such compositions may contain encapsulated NMDA antagonist such that a plasma concentration of greater than about 10 ng/ml of NMDA antagonist is maintained for up to about 60 days, up to about 30 days, or up to about 21 days. In particular embodiments, the formulation may include about 200 mg to about 500 mg of encapsulated NMDA antagonist. Further embodiments are directed to methods for treating depression by administering to a patient in need of treatment a formulation of NMDA antagonist containing encapsulated NMDA antagonist.

“NMDA antagonists” include various compounds including competitive antagonists, non-competitive antagonists, non-competitive channel blockers, and glycine antagonists. Such compounds are well-known in the art, and NMDA antagonists of each class are encompassed by the invention. In some embodiments, the NMDA antagonist may be a non-competitive channel blocker such as, for example, minocycline, amantadine, atomoxetine, AZD6765, agmatine, chloroform, dextrallorphan, dextromethorphan, dextrorphan, diphenidine, dizocilpine (MK-801), ethanol, eticyclidine, gacyclidine, ketamine, magnesium, memantine, methoxetamine, nitromemantine, nitrous oxide, PD-137889, phencyclidine, rolicyclidine, methoxydine, tiletamine, neramexane, eliprodil, etoxadrol, dexoxadrol, WMS-2539, NEFA, remacemide, delucemine, 8A-PDHQ, and the like and combinations thereof. In certain embodiments, the NMDA agonist may be ketamine (2-(2-chlorophenyl)-2-(methylamino)-cyclohexanone), S-ketamine, esketamine, R-ketamine, racemic ketamine, norketamine (2-(2-chlorophenyl)-2-amino-cyclohexanone), S-norketamine, R-norketamine, and racemic norketamine. Ketamine is typically a racemic mixture of S- and R-ketamine, although S-ketamine has been found recently to be twice as potent as R-ketamine and to allow faster recovery with fewer negative side effects than the racemic mixture.

The term “encapsulated NMDA antagonist” refers to NMDA antagonist, in free base, salt, or hydrate form, is encapsulated in a microparticle or nanoparticle. Embodiments are not limited to particular types of microparticles or nanoparticles. For example, in certain embodiments, an active agent may be encapsulated in microparticles made from biodegradable polymers, such as polylactides (PLA), polyglycolides (PGA), and poly(lactide-co-glycolide) (PLGA) polymers. In some embodiments, the microparticles may also include derivatives of PLA or PGA, such as poly butylene succinate (PBS), polyhydroxyalkanoate (PHA), polycaprolactone acid lactone (PCL), polyhydroxybutyrate (PHB), glycolic amyl (PHV), PHB and PHV copolymer (PHBV), and poly lactic acid (PLA)-polyethylene glycol (PEG) copolymers (PLEG). PLA/PGA/PLGA degrade in the body by simple hydrolysis of the ester backbone to non-harmful and non-toxic compounds. The in vivo degradation products are either excreted by the kidneys or eliminated as carbon dioxide and water through well-known biochemical pathways. Typically, the active agent can be entrapped in solid microparticles in which release of the agent is achieved by either bioerosion of the microparticles or diffusion out of the microparticle.

For purposes of this disclosure reference to a single biodegradable is meant to encompass the other biodegradable polymers. For example, the term “PLGA microparticle” as used herein below is meant as example biodegradable polymer, and is meant to encompass microparticle composed of PLGA as well as microparticles composed of PLA, PGA, PBS, PHA, PCL, PHB, PHV, PHBV, PEG, PLEG, and copolymers thereof.

The molecular weight of the biodegradable polymer units that make up the microparticles can affect the rate of degradation of the microparticle and subsequent release of the drug. For example, microparticles composed of polymer units having low molecular weights generally degrade faster and release the drug at an earlier period when compared to microparticles composed of polymer with high molecular weight polymer units. In various embodiments, the microparticles may be composed of polymer units having molecular weights of from about 5 kiloDalton (kDa) to about 150 kDa, about 5 kDa to about 125 kDa, about 10 kDa to about 100 kDa, about 15 kDa to about 75 kDa, or about 20 kDa to about 50 kDa, or any individual molecular weight or range encompassed by these example ranges. Specific examples include about 5 kDa, about 10 kDa, about 15 kDa, about 20 kDa, about 25 kDa, about 30 kDa, about 40 kDa, about 50 kDa, about 60 kDa, about 75 kDa, about 100 kDa, about 150 kDa, and ranges between any of these example values.

In some embodiments, the ratio of biodegradable polymer components, for example, PLA to PGA, in the microparticles can be about 1:99 to about 99:1 by weight, about 10:90 to about 90:10 by weight, about 30:70 to about 70:30 by weight, about 40:60 to about 60:40 by weight, about 50:50 by weight, or about 30:70 to about 40:60 by weight. Specific examples of ratio of PLA to PGA include about 30:70 by weight, about 40:60 by weight, 50:50 by weight, about 60:40 by weight, about 70:30 by weight, about 75:25 by weight, and ranges between any two of these values. Microparticles having a higher concentration of lactide units degrade more slowly allowing for delayed release of the active agent.

Microparticles of encapsulated NMDA antagonist can have a mean particle diameter (MPD) of about 0.5 micrometers (μm) to about 120 μm, about 1 μm to about 100 μm, about 2 μm to about 75 μm or any range or individual value encompassed by these example ranges. In particular embodiments, the mean particle diameter of the microparticles may be about 30 μm to about 100 μm, about 35 μm to about 75 μm, about 40 μm to about 60 μm, or any individual diameter or range encompassing these example ranges. In various embodiments, the microparticles may have a monomodal particle size distribution in which a single maxima discernable on a particle size distribution curve (weight percent or population on the ordinate or Y-axis, and particle size/diameter on the abscissa or X-axis). In some embodiments, the microparticles may have a monodisperse particle size distribution, meaning all of the particles have substantially the same mass.

In various embodiments, the microparticles may have an NMDA antagonist loading of about 0.5 wt. % to about 50 wt. %, about 1 wt. % to about 40 wt. %, about 10 wt. % to about 35 wt. %, about 15 wt. % to about 25 wt. %, or any range or individual value encompassed by these ranges. “Drug (e.g. NMDA antagonist) loading” as used herein is defined as the weight of drug in the final microparticle formulation divided by the weight of microparticles in the final formulation (units of % w/w; e.g. weight naloxone/weight PLGA).

The biodegradable polymer components of the microparticles, for example, PLGA, may be capped or uncapped. The term “uncapped PLGA” indicates that the PLGA of the microparticles described or its underlying components, PLA and PGA, have not been functionalized. Thus, “uncapped PLGA” or “uncapped microparticles” contain carboxyl (—COOH) end groups at polymer component termini. The term “capped PLGA” or “capped microparticles” indicates that the PLGA of the microparticles described or its underlying components, PLA and PGA, have been functionalized. For example, the carboxyl end groups of “capped PLGA” have undergone a chemical reaction, i.e. functionalization, to produce, for example, ester end groups (—COOR). Without wishing to be bound by theory, capped PLGA may be less charged and, therefore, less likely to produce ionic interactions with free NMDA antagonists. The use of capped PLGA in microparticles may reduce or eliminate any delay in immediate release of NMDA antagonist upon administration of the formulations of embodiments of the invention.

The microparticle encapsulation of NMDA antagonists can be performed by any means, including but not limited to, microemulsion and spray drying methods. For example, PLGA polymers of known molecular weights can be dissolved in organic solvents, such as halogenated hydrocarbons such as methylene chloride, chloroform, and carbon tetrachloride; aromatic hydrocarbons such as toluene and xylene; or mixtures or combinations thereof. The NMDA antagonists may be dissolved in an aqueous solvent, such as polyvinyl alcohol, polyvinyl pyrrolidone, carboxymethyl cellulose, lecithin, and gelatin, and the PLGA solution and the anticancer agent solution can be mixed and sonicated to form a uniform distribution of the NMDA antagonists and the PLGA polymer. Homogenization is subsequently performed to form the polymer particle emulsion. The resulting polymer emulsion is stirred until the organic solvent is evaporated, resulting in precipitation of polymer particles that encapsulate the NMDA antagonists. The size of the microparticles may be controlled by varying homogenization speed during emulsification. Encapsulation of NMDA antagonists can also be achieved by the spray coating (hereby referred to as ‘core-shell’) method. In this instance, a NMDA antagonist drug core is encapsulated by a polymeric shell to form a microparticle. Characteristics of this polymer shell can then be altered to achieved desired release characteristics.

The

The pharmaceutical compositions disclosed herein provide for sustained release of NMDA antagonist for a time period of about 5 days to 60 days, 5 days to 30 days, 5 days to 21 days or any range or individual term encompassed by these example ranges. “Sustained release” refers to the process in which the NMDA antagonist is released gradually over a period of time.

Further embodiments include compositions containing NMDA antagonists dissolved in oils producing a sustained release formulation. In such embodiments, the NMDA antagonists may ionically associate with the oils without forming microparticles or nanoparticles. Without wishing to be bound by theory, this ionic association may delay release of the NMDA antagonist after administration as the oil is broken down releasing additional NMDA antagonists over time. The oil used in such embodiments is not limited, for example, the oil may vegetable oil, olive oil, grapeseed oil, tea tree oil, almond oil, avocado oil, sesame oil, evening primrose oil, sunflower oil, kukui nut oil, jojoba oil, walnut oil, peanut oil, pecan oil, macadamia nut oil, coconut oil, and the like and combinations thereof. The amount of NMDA antagonist in such embodiments may be from about 100 mg to about 500 mg, or any range or individual amount encompassed by this range, and the oil may make up the remaining volume of the composition. In some embodiments, the NMDA antagonist may be a free base form of the NMDA antagonist, for example, ketamine free base or ketamine free base, which may render the NMDA antagonist more hydrophobic and more soluble in the oil than salt forms of the NMDA antagonists.

### Hydrogel

The sustained release pharmaceutical compositions disclosed herein may further contain a hydrogel. The hydrogel may help to hold the microparticles in the composition without clumping, maintain the integrity of the microparticles by buffering the pH, and aid in administration of the composition. Non-limiting examples of hydrogels include methyl cellulose (MC), ethyl cellulose (EC), ethyl methyl cellulose (EMC), hydroxyethyl cellulose (HEC), hydroxylpropyl cellulose (HPC), hydroxymethyl cellulose (HMC), hydroxypropylmethyl cellulose (HPMC), ethylhydroxyethyl cellulose (EHEC), hydroxyethylmethy cellulose (HEMC), methylhydroxyethyl cellulose (MHEC), methylhydroxypropylcellulose (MHPC), and hydroxyethylcarboxymethyl cellulose (HECMC).

Other materials that can be used to form a hydrogel include modified alginates. Alginate is a carbohydrate polymer isolated from seaweed that can be crosslinked to form a hydrogel by exposure to a divalent cation, such as calcium. Additionally, polysaccharides that gel by exposure to monovalent cations, including bacterial polysaccharides, such as gellan gum, and plant polysaccharides, such as carrageenans, may be crosslinked to form a hydrogel, using methods known in the art. Tragacanth, pectin, guar gum, xanthan gum, and polyacrylamide may also be used as hydrogels.

In some embodiments, the formulations discussed above may have an inherent viscosity of about 300 cP or less, about 200 cP or less, about 100 cP or less or about 50 cP or less. Combinations of viscosity reducing agents may be used to achieve the desired viscosity. For example, polyethylene glycol polymers, surfactants, organic solvents, aqueous solvents and combinations thereof are suitable for use as viscosity reducing agents. The amount of viscosity reducing agent present in the sustained release composition can range from about 5 wt % to about 40 wt % of the total weight of the sustained release composition.

### Formulations

The pharmaceutical compositions of the invention are typically used in the form of a drug reservoir such as injectable microparticles, passive transdermal/transmucosal drug delivery or electrotransport drug delivery systems. It will be appreciated by those skilled in the art that the inventive formulations described herein can be combined with suitable carriers to prepare alternative drug dosage forms (e.g., oral capsule, topical ointment, rectal and/or vaginal suppositories, buccal patches, or an aerosol spray).

It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

Pharmaceutical compositions disclosed herein can include suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols. In some embodiments, the pharmaceutical excipient may include, without limitation, binders, coating, disintegrants, fillers, diluents, flavors, colors, lubricants, glidants, preservatives, sorbents, sweeteners, conjugated linoleic acid (CLA), gelatin, beeswax, purified water, glycerol, any type of oil, including, without limitation, fish oil or soybean oil, or the like.

In some embodiments, the pharmaceutical composition may include one or more disintegrant component, such as croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the pharmaceutical composition may include one or more diluent component, such as mannitol, lactose, sucrose, maltodextrin, sorbitol, xylitol, powdered cellulose, microcrystalline cellulose, carboxymethyl-cellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the pharmaceutical composition may include one or more optional lubricant component, such as stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

### Administration

Disclosed herein are methods for treating neuropsychiatric disorders in a subject. In various embodiments, such methods may include the step of administering a therapeutically effective amount of a formulation disclosed herein. Neuropsychiatric disorders include various disorders including, for example, schizophrenia, bipolar disorder, Alzheimer's disease, Parkinson's disease, dementia, ataxia, spinocerebellar degeneration, attention deficit disorder (ADD), attention deficit, hyperactivity disorder (ADHD), depression, and mild cognitive impairment.

The term “subject” includes animals which can be treated using the methods of the invention. Examples of animals include mammals, such as mice, rabbits, rats, horses, goats, dogs, cats, pigs, cattle, sheep, and primates (e.g. chimpanzees, gorillas, and, preferably, humans). In a further embodiment, the patient is a cancer patient, e.g., a human suffering from cancer, a tumor or tumors.

In certain embodiments, methods may be directed to treating depression. “Depression” refers to a clinical syndrome that includes a persistent sad mood or loss of interest in activities, which lasts for at least two weeks in the absence of treatment. The DSM-IV criteria can be used to diagnose patients as suffering from depression. Symptoms of depression include, for example, feelings of sadness, emptiness, anxiety, helplessness, worthlessness, guilt, or hopelessness, irritability or crankiness, loss of interest in activities, such as hobbies or sex, loss of energy, extreme tiredness, trouble concentrating, trouble remembering details, feeling overwhelmed by decisions, changes in sleep patterns, changes in appetite, weight gain or weight loss, aches and pains, headaches, cramps, upset stomach, digestive problems, and the like and combinations thereof.

In some embodiments, methods may be directed to treating bipolar disorder. Bipolar disorder or manic-depressive disorder (also referred to a bipolarism or manic depression) is a psychiatric diagnosis that describes a category of mood disorders defined by the presence of one or more episodes of abnormally elevated mood clinically referred to as mania or, if milder, hypomania. Individuals who experience manic episodes also commonly experience depressive episodes or symptoms, or mixed episodes in which features of both mania and depression are present at the same time. These episodes are usually separated by periods of “normal” mood, but in some individuals, depression and mania may rapidly alternate, known as rapid cycling. Extreme manic episodes can sometimes lead to psychotic symptoms such as delusions and hallucinations. The disorder has been subdivided into bipolar I, bipolar II, cyclothymia, and other types, based on the nature and severity of mood episodes experienced; the range is often described as the bipolar spectrum. Patients can be diagnosed as having bipolar disorder using the DSM-IV criteria.

In particular embodiments, methods may be directed to treating Asperger's syndrome. Asperger's syndrome is an autism spectrum disorder, and people with it therefore show significant difficulties in social interaction, along with restricted and repetitive patterns of behavior and interests. It differs from other autism spectrum disorders by its relative preservation of linguistic and cognitive development. Although not required for diagnosis, physical clumsiness and atypical use of language are frequently reported. Patients can be diagnosed as suffering from Asperger's disorder by using the DSM-IV criteria.

In some embodiments, methods may be directed to treating ADD or ADHD. The term ADD and ADHD refer to disorders that are commonly exhibited by children, characterized by increased motor activity and a decreased attention span. The DSM-IV criteria can be used to diagnose attention deficit disorder.

Administration can be systemic, parenteral, topical, or oral. For example, administration can be, but is not limited to, parenteral, subcutaneous, intravenous, intramuscular, intraperitoneal, transdermal, oral, buccal, ocular routes, or intravaginally, by inhalation, by depot injections, or by implants. In particular embodiments, administering can be carried out by injection including, for example, depo injection, intramuscular injection, or subcutaneous injection, and the like, or by oral, sublingual, or intranasal administration, and the like. The selection of the specific route of administration and the dose regimen is to be adjusted or titrated by the clinician according to methods known to the clinician in order to obtain the optimal clinical response. The amount of compounds to be administered is that amount which is therapeutically effective. The dosage to be administered will depend on the characteristics of the subject being treated, e.g., the particular animal or human being treated, age, weight, health, types of concurrent treatment, if any, and frequency of treatments, and can be easily determined by one of skill in the art (e.g., by the clinician).

In certain embodiments, the formulations of embodiments may be administered by a syringe. The sustained release composition is formulated so that the composition can be readily implanted (e.g., by injection) into the desired location to form a mass that can remain in place for the period suitable for controlled release of the NMDA antagonist. The mechanical and rheological properties suitable for injectable depot compositions are known in the art. Typically, the polymer of the depot vehicle with particulates are present in an appropriate amount of solvent such that the depot composition can be so implanted.

For oral administration, the pharmaceutical composition can be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

For oral administration, the hydrogel formulation is preferably encapsulated by a retardant coating, e.g., a bioerodible polymer. Upon dissolution or erosion of the encapsulating material, the hydrogel core becomes exposed and the drug contained within the gel can be released for enteric adsorption. Bioerodible coating materials may be selected from a variety of natural and synthetic polymers, depending on the agent to be coated and the desired release characteristics. Exemplary coating materials include gelatins, carnauba wax, shellacs, ethylcellulose, cellulose acetate phthalate or cellulose acetate butyrate. Release of the agent is controlled by adjusting the thickness and dissolution rate of the polymeric coat.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For intranasal administration, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compositions of the present invention can also be formulated in rectal compositions such as suppositories or retention enemas, e.g., containing conventional suppository bases such as cocoa butter or other glycerides.

In transdermal administration, the compositions of the present invention, for example, can be applied to a plaster, or can be applied by transdermal, therapeutic systems that are consequently supplied to the organism. In some embodiments, the formulation can be delivered using microneedle apparatuses.

### Packs and Kits

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers having therapeutically or prophylactically effective amounts of the sustained release compositions in pharmaceutically acceptable form. The sustained release compositions in a vial of a kit of the invention may be in the form of a pharmaceutically acceptable solution, e.g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e.g., saline, dextrose solution, etc.), preferably sterile, to reconstitute the complex to form a solution for injection purposes.

In another embodiment, a kit of the invention further comprises a needle or syringe, preferably packaged in sterile form, for injecting the complex, and/or a packaged alcohol pad. Instructions are optionally included for administration of sustained release compositions by a clinician or by the patient. Such kits may contain one or more vial of a sustained release NMDA antagonist formulation that is manually loaded into a syringe before administering or autoinjectors that are pre-loaded with the formulation in an appropriate amount for administration.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1

Dried norketamine loaded PLGA microparticles having the following compositions were prepared.

TABLE 1

| | Input Polymer (g) | Input drug (g) | Yield |
|---|---|---|---|
| AP073 | 0.5 | 0.1 | 35% |
| AP081 | 0.5 | 0.15 | 15% |
| AP126 | 0.5 | 0.15 | 45% |

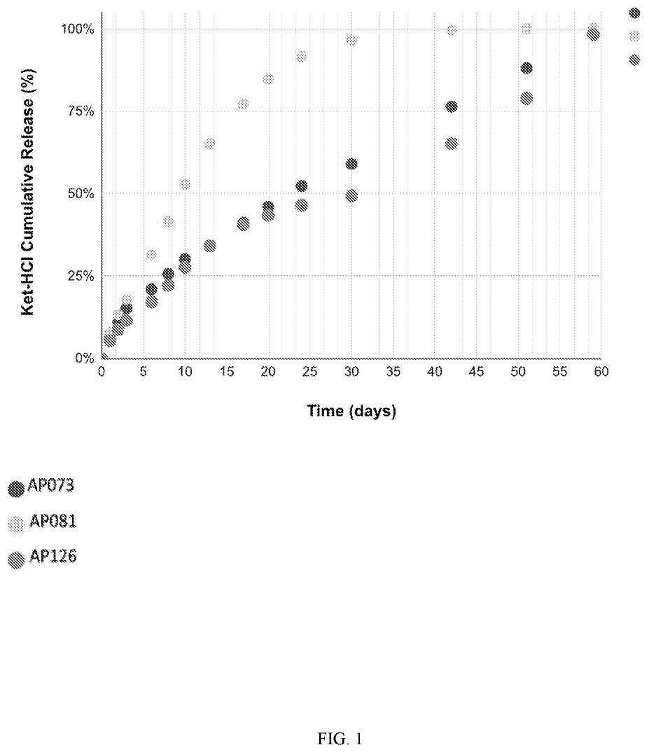

The microparticles were suspended in PBS and centrifuged to produce a pellet of microparticles. Supernatant was extracted from vials are regular intervals and the concentration of norketamine in the supernatant was determined using UV plate reader at 220 nm. Cumulative release data is presented in FIG. 1.

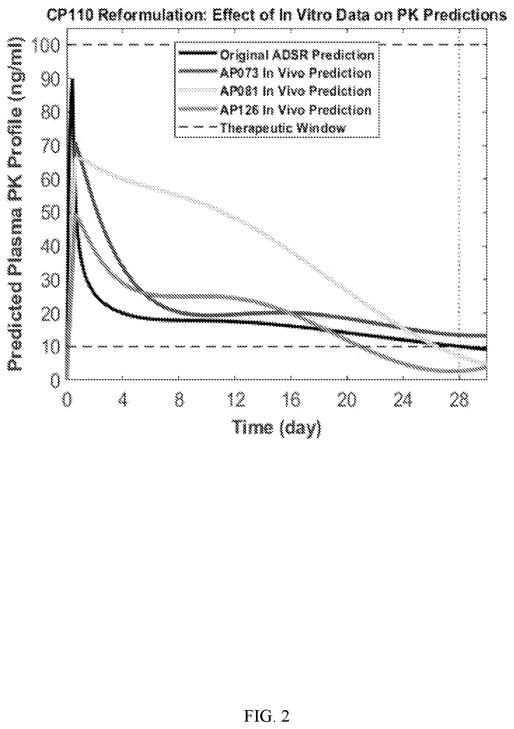

Predicted plasma PK profiles based on this release data for each example formulation are plotted in FIG. 2, versus the original predicted release profile (Original ADSR Prediction). These data show a sustained release of norketamine within the therapeutic window for up to 20 days and as long as 28 days.

1. A composition comprising about 100 mg to about 500 mg of NMDA antagonist encapsulated in microparticles and a pharmaceutically acceptable excipient, diluent, or carrier

2. The composition of claim 1, wherein the NMDA antagonist is selected from the group consisting of minocycline, amantadine, atomoxetine, AZD6765, agmatine, chloroform, dextrallorphan, dextromethorphan, dextrorphan, diphenidine, dizocilpine (MK-801), ethanol, eticyclidine, gacyclidine, ketamine, magnesium, memantine, methoxetamine, nitromemantine, nitrous oxide, PD-137889, phencyclidine, rolicyclidine, methoxydine, tiletamine, neramexane, eliprodil, etoxadrol, dexoxadrol, WMS-2539, NEFA, remacemide, delucemine, 8A-PDHQ, and combinations thereof.

3. The composition of claim 1, wherein the NMDA antagonist is selected from the group consisting of ketamine (2-(2-chlorophenyl)-2-(methylamino)-cyclohexanone), S-ketamine, esketamine, R-ketamine, racemic ketamine, norketamine (2-(2-chlorophenyl)-2-amino-cyclohexanone), 5-norketamine, R-norketamine, and racemic norketamine.

4. The composition of claim 1, wherein the encapsulated NMDA comprises microparticles composed of a biodegradable polymer selected from the group consisting of polylactides (PLA), polyglycolides (PGA), poly(lactide-co-glycolide) (PLGA) polymers, poly butylene succinate (PBS), polyhydroxyalkanoate (PHA), polycaprolactone acid lactone (PCL), polyhydroxybutyrate (PHB), glycolic amyl (PHV), PHB and PHV copolymer (PHBV), and poly lactic acid (PLA)-polyethylene glycol (PEG) copolymers (PLEG), and copolymers thereof.

5. The composition of claim 2, wherein the poly(lactide-co-glycolide) (PLGA) is capped.

6. The composition of claim 2, wherein the poly(lactide-co-glycolide) (PLGA) comprises a ratio of polylactides (PLA) to polyglycolides (PGA) of 50:50 by weight to about 60:40 by weight.

7. The composition of claim 1, wherein the microparticles have a mean particle diameter of about 40 μm to about 60 μm.

8. The composition of claim 1, further comprising methyl cellulose (MC), ethyl cellulose (EC), ethyl methyl cellulose (EMC), hydroxyethyl cellulose (HEC), hydroxylpropyl cellulose (HPC), hydroxymethyl cellulose (HMC), hydroxypropylmethyl cellulose (HPMC), ethylhydroxyethyl cellulose (EHEC), hydroxyethylmethy cellulose (HEMC), methylhydroxyethyl cellulose (MHEC), methylhydroxypropylcellulose (MHPC), hydroxyethylcarboxymethyl cellulose (HECMC), and combinations thereof.

9. The composition of claim 1, wherein the excipient is selected from the group consisting of calcium carbonate, calcium phosphate, various sugars, starches, cellulose derivatives, gelatin, polyethylene glycol, and combinations thereof.

10. The composition of claim 1, further comprising a binder, coating, disintegrant, filler, diluent, flavor, color, lubricant, glidant, preservative, sorbent, sweetener, conjugated linoleic acid (CLA), gelatin, beeswax, purified water, glycerol, pharmaceutically acceptable oils, and combinations thereof.

11. A composition comprising about 100 mg to about 500 mg of NMDA antagonist and a pharmaceutically acceptable oil.

12. The composition of claim 11, wherein the pharmaceutically acceptable oil is selected from the group consisting of vegetable oil, olive oil, grapeseed oil, tea tree oil, almond oil, avocado oil, sesame oil, evening primrose oil, sunflower oil, kukui nut oil, jojoba oil, walnut oil, peanut oil, pecan oil, macadamia nut oil, coconut oil, and combinations thereof.

13. The composition of claim 11, wherein the NMDA antagonist is ionically associated with the pharmaceutically acceptable oil.

14. The composition of claim 11, wherein the NMDA antagonist is selected from the group consisting of minocycline, amantadine, atomoxetine, AZD6765, agmatine, chloroform, dextrallorphan, dextromethorphan, dextrorphan, diphenidine, dizocilpine (MK-801), ethanol, eticyclidine, gacyclidine, ketamine, magnesium, memantine, methoxetamine, nitromemantine, nitrous oxide, PD-137889, phencyclidine, rolicyclidine, methoxydine, tiletamine, neramexane, eliprodil, etoxadrol, dexoxadrol, WMS-2539, NEFA, remacemide, delucemine, 8A-PDHQ, and combinations thereof.

15. A method for treating neuropsychiatric disorders comprising administering to a subject in need of treatment a composition about 100 mg to about 500 mg of NMDA antagonist encapsulated in microparticles and a pharmaceutically acceptable excipient, diluent, or carrier wherein the composition releases the NMDA antagonist for about 7 days to about 90 days.

16. The method of claim 15, wherein administering is carried out parenteral injection, subcutaneous injection, intravenous injection, intramuscular injection, intraperitoneal injection, transdermally, orally, buccally, ocularly, intravaginally, by inhalation, by depot injections, or by implants.

17. The method of claim 15, wherein administering is carried out by depo injection, intramuscular injection, or subcutaneous injection.

18. The method of claim 15, wherein the NMDA antagonist is selected from the group consisting of minocycline, amantadine, atomoxetine, AZD6765, agmatine, chloroform, dextrallorphan, dextromethorphan, dextrorphan, diphenidine, dizocilpine (MK-801), ethanol, eticyclidine, gacyclidine, ketamine, magnesium, memantine, methoxetamine, nitromemantine, nitrous oxide, PD-137889, phencyclidine, rolicyclidine, methoxydine, tiletamine, neramexane, eliprodil, etoxadrol, dexoxadrol, WMS-2539, NEFA, remacemide, delucemine, 8A-PDHQ, and combinations thereof.

19. The method of claim 15, wherein the encapsulated NMDA comprises microparticles composed of a biodegradable polymer selected from the group consisting of polylactides (PLA), polyglycolides (PGA), poly(lactide-co-glycolide) (PLGA) polymers, poly butylene succinate (PBS), polyhydroxyalkanoate (PHA), polycaprolactone acid lactone (PCL), polyhydroxybutyrate (PHB), glycolic amyl (PHV), PHB and PHV copolymer (PHBV), and poly lactic acid (PLA)-polyethylene glycol (PEG) copolymers (PLEG), and copolymers thereof.

20. The method of claim 15, wherein the poly(lactide-co-glycolide) (PLGA) comprises a ratio of polylactides (PLA) to polyglycolides (PGA) of 50:50 by weight to about 60:40 by weight.

21. The method of claim 15, wherein the microparticles have a mean particle diameter of about 40 μm to about 60 μm.
